(19) United States
(12) Patent Application Publication
(10) Pub. No.: US 2017/0044090 A1
(43) Pub. Date: Feb. 16, 2017

(54) Ring Opening Of Epoxidized Fatty Acid Esters

(71) Applicant: Solazyme, Inc., South San Francisco, CA (US)

(72) Inventors: Herman P. Benecke, Columbus, OH (US); Jeffrey T. Cafmeyer, Columbus, OH (US); Daniel B. Garbark, Blacklick, OH (US)

(73) Assignee: Solazyme, Inc., South San Francisco, CA (US)

(21) Appl. No.: 15/306,363

(22) PCT Filed: Oct. 29, 2014
(86) PCT No.: PCT/US2014/062821

§ 371 Date: Oct. 24, 2016

Related U.S. Application Data
(63) Continuation-in-part of application No. 14/262,070, filed on Apr. 25, 2014, now Pat. No. 9,249,252.
(60) Provisional application No. 61/816,648, filed on Apr. 26, 2013.
(60) Provisional application No. 61/831,571, filed on Jun. 5, 2013.
(60) Provisional application No. 61/887,278, filed on Oct. 4, 2013.
(60) Provisional application No. 61/942,524, filed on Feb. 20, 2014.

(51) Int. Cl. C07C 69/675; C08G 18/36

(52) U.S. Cl. CPC C07C 69/675; C08G 18/36

(57) ABSTRACT

This invention relates to a ring opening method. The method includes reacting an epoxidized fatty acid ester with an alcohol to open an epoxy ring of the ester. A salt of tetrafluoroboric acid is used as a catalyst in the reaction. In another embodiment, the invention relates to a method of producing a polyurethane. An epoxidized fatty acid ester is reacted with an alcohol to open an epoxy ring of the ester, thereby to produce a polyol. A salt of tetrafluoroboric acid is used as a catalyst in the reaction. The polyol is then reacted with an isocyanate to produce a polyurethane. In a further embodiment, the polyol is used to produce a product selected from polyurethanes, coatings, adhesives, sealants, elastomers and lubricants.

### CROSS-REFERENCE TO RELATED APPLICATIONS

This application is a continuation-in-part patent application of U.S. patent application Ser. No. 14/262,070, filed Apr. 25, 2014, and this application is a continuation-in-part patent application of International Application No. PCT/US14/35476, filed Apr. 25, 2014. Ser. No. 14/262,070 and PCT/US14/35476 claim the benefit of U.S. Provisional Patent Application No. 61/816,648, filed Apr. 26, 2013; 61/831,571, filed Jun. 5, 2013; 61/887,278, filed Oct. 4, 2013; and 61/942,524, filed Feb. 20, 2014. The disclosures of all of these applications are incorporated herein by reference.

### BACKGROUND OF THE INVENTION

Products derived from renewable feedstocks such as vegetable, algal or animal oils or fats would be desirable to help reduce the world dependence on oil and other fossil-derived hydrocarbons. Moreover, these oils or fats are typically biodegradable, which would help reduce the introduction of waste into the environment. Products produced with these feedstocks may also have improved properties.

An important way to utilize the renewable feedstocks is by the production of polyols from the feedstocks which can be used to produce a number of products. For example, there has been great interest in the ring opening of epoxidized soybean oil and other triglyceride oils for mono-ol and di-ol polyol functionalization at the original olefin site. The main issues in the processes have been undesired side reactions (e.g., polymerization, intramolecular ether formation, and hydrolysis of the triglyceride) and difficult purifications leading to decreased yields of desired products.

Therefore, it would be desirable to provide a method for the production of polyols that overcomes some of the problems of the earlier methods.

### SUMMARY OF THE INVENTION

This invention relates to a ring opening method. The method comprises reacting an epoxidized fatty acid ester with an alcohol to open an epoxy ring of the ester. A salt of tetrafluoroboric acid is used as a catalyst in the reaction.

In another embodiment, the invention relates to a method of producing a polyurethane. An epoxidized fatty acid ester is reacted with an alcohol to open an epoxy ring of the ester, thereby to produce a polyol. A salt of tetrafluoroboric acid is used as a catalyst in the reaction. The polyol is then reacted with an isocyanate to produce a polyurethane.

In a further embodiment, the invention relates to a method of producing a product. An epoxidized fatty acid ester is reacted with an alcohol to open an epoxy ring of the ester, thereby to produce a polyol. A salt of tetrafluoroboric acid is used as a catalyst in the reaction. The polyol is then used to produce a product selected from polyurethanes, coatings, adhesives, sealants, elastomers and lubricants.

Various aspects of this invention will become apparent to those skilled in the art from the following detailed description of the preferred embodiment.

### DETAILED DESCRIPTION OF THE PREFERRED EMBODIMENT

The present invention relates to a ring opening method. The method comprises reacting an epoxidized fatty acid ester with an alcohol to open an epoxy ring of the ester. A salt of tetrafluoroboric acid is used as a catalyst in the reaction.

The fatty acid ester can be any type which can be epoxidized to produce an ester including an epoxy ring. For example, the fatty acid ester may be a triglyceride, a fatty acid methyl ester or a fatty acid ethyl ester.

Triglycerides, also known as triacylglycerols or triacylglycerides, are the major component of plant oils, algal oils and animal fats (the terms “oils” and “fats” are used interchangeably). Triglycerides can also be produced synthetically. A triglyceride is a compound consisting of three fatty acids esterified to a glycerol. Some examples of typical fatty acids of plant oils are stearic, palmitic, oleic, linoleic and linolenic.

Plant oils (or vegetable oils) are any of a large group of oils obtained from the seeds, fruits or leaves of plants. Some nonlimiting examples of plants from which oils can be derived include soybean, canola, palm, olive, peanut, sesame, sunflower and safflower.

Many different types of algae produce triglyceride oils. Specific examples include heterotrophic or obligate heterotrophic microalgae of the phylum Chlorophtya, the class Trebouxiophytae, the order Chlorellales, or the family Chlorellacae. Other examples of oleaginous microalgae and methods of cultivation are provided in Published PCT Patent Applications WO2008/151149, WO2010/06032, WO2011/150410, WO2011/150411, WO2012/061647, and WO2012/106560.

In certain embodiments, the triglyceride is selected from the group consisting of vegetable oils, animal fats and combinations thereof. In other embodiments, the triglyceride excludes algal oils.

A number of chemical manufacturers sell different types of epoxidized fatty acid esters, most commonly epoxidized soybean oil. Alternatively, the epoxidation of the fatty acid ester can be included as part of the present invention. For example, in a typical epoxidation reaction a triglyceride is exposed to an oxidant such as a peroxyacid or hydroperoxide. Examples of peroxyacid that may be used include peroxyformic acid, peroxyacetic acid, trifluoroperoxyacetic acid, benzyloxyperoxyformic acid, 3,5-dinitroperoxybenzoic acid, m-chloroperoxybenzoic acid, and combinations thereof. The peroxy acid may be added directly to the reaction, or may be formed in-situ by reacting a hydroperoxide compound with an acid such as formic acid, benzoic acid, acetic acid or fatty acids such as oleic acid. Examples of typical hydroperoxides that may be utilized include hydrogen peroxide, tert-butylhydroperoxide, triphenysilylhydroperoxide, cumylhydroperoxide, and combinations thereof.

In the ring opening reaction, the epoxidized fatty acid ester is reacted with an alcohol to open the epoxy ring of the ester. The resulting product is often a polyol. In some embodiments, the product may be further reacted to produce a polyol. Any suitable type of alcohol, or combinations of different alcohols, may be used. In certain embodiments, monohydric alcohols having between one and six carbon atoms are utilized, more particularly one to four carbon atoms, further more particularly methanol, ethanol or propanol. The alcohol reactant may also be a polyol. For instance, polyols having two or less hydroxyl groups per molecule may be used. Examples of polyols include ethylene glycol, propylene glycol, 1,3 propanediol, buylene-glycol, 1,4-butane diol, 1,5-pentanediol, 1,6-hexanediol, polyethylene glycol and polypropylene glycol, and vegetable oil-based polyols (for example, polyols as described in U.S. Pat. No. 6,433,121). In certain embodiments, the reaction with propylene glycol results in a desirably high hydroxyl value.

The ring opening reaction is catalyzed using a salt of tetrafluoroboric acid (H3OBF4). In certain embodiments, the salt is a metal salt. For example, the catalyst may be copper tetrafluoroborate or iron tetrafluoroborate. The catalyst is used in any suitable amount. For example, the catalyst may be present in an amount ranging from 0.01 wt % to 0.3 wt %, preferably from about 0.05 wt % to 0.15 wt %, based on the total weight of the reaction mixture.

A reaction schematic for the production of a polyol from an epoxidized oleic acid ester (one of many fatty acids contained in soybean oil) can be seen below:

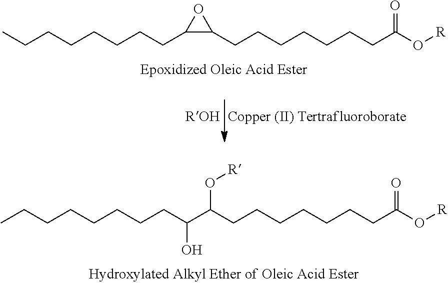

A number of problems can be overcome by the use of a tetrafluoroborate salt as the catalyst instead of tetrafluoroboric acid. For example, two issues can arise when tetrafluoroboric acid is used to catalyze the reaction. First, tetrafluoroboric acid causes methanol to transesterify the triglyceride of soybean oil readily forming fatty acid methyl ester which are not desired. This transesterification is difficult to control due to the reaction exotherm and reaction conditions. Second, the purification of the product polyol requires base treatment and leads to emulsions and product loss. In contrast, when the tetrafluoroborate salt is used as a catalyst, the ring opening of epoxide readily takes place and there is little to no transesterification of the triglyceride backbone. Furthermore, the final polyol hydroxyl values are near theoretical. In certain embodiments, the hydroxyl value is within a range of from about 75 to about 400. The hydroxyl value can be measured by any suitable method; for example, by ASTM D 1957 or ASTM E-222-10. The reaction has been run with and without a co-solvent and has produced nearly identical product polyols.

The tetrafluoroborate salt also can overcome problems with the use of strong acids as catalysts. For example, a problem with performing this reaction with strong acid is that the epoxides of the major fatty acid component in soybean oil (linoleic acid) were found to form a significant amount of a tetrahydrofuranal structure via intra-molecular etherification or inter-molecular polymerizing rather than simple ring opening to the diol. This self-etherification/polymerization leads to an undesired product with increased molecular weight and viscosity. Also, the reaction conditions were leading to hydrolysis of the triglyceride backbone producing mono- and di-glycerides. Using a metal salt of tetrafluoroboric acid, for example copper tetrafluoroborate, we sought to activate the ring opening of the oxirane while minimizing the side-reactions due to less acidic form of the catalyst. In addition, by using the metal salt we also avoid the need to neutralize the acid during the work up procedure of the product. The metal salt is very soluble in water and therefore can be removed from the polyol with simple water or brine washes. The present method can produce the desired products in high yield.

In another embodiment, the polyol produced by the ring opening method can be used to produce a lubricant. For example, the following schematic shows a soybean oil based lubricant that can be produced according to the invention.

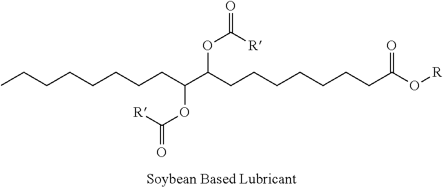

In another embodiment, the polyol produced by the ring opening method can be used to produce a polyurethane. A polyurethane is a compound that comprises a carbamate (urethane) linkage. In some embodiments the polyurethane is a polymer of organic units. The polymer is prepared by the reaction of a first organic unit comprising an isocyanate moiety and a second organic unit comprising a hydroxyl group or an epoxide group. In the present invention, the polyol produced by the ring opening method can function as the second organic unit comprising a hydroxyl group. In certain embodiments, the polyol is subjected to an isocyanate condensation reaction to form the polyurethane. The polyurethane can be a foam or a denser product.

The polyurethane can also be prepared with and/or contain additives, alcohols, blowing agents, fillers and/or surfactants. Additives include release agents, anti-oxidants, flame retardant additives, cell size control agents, cell opening agents, colorants, preservatives, static dissipative agents, plasticizers, and crosslinking agents. Blowing agents include low temperature boiling point solvents for generating gases to expand the polyurethane foam during its synthesis. In some embodiments the surfactant is a silicon surfactant.

Methods and components for producing polyurethanes can also be found in US 2005/0282921 which is incorporated by reference herein.

Polyurethanes are used in many different applications including both flexible and rigid materials. Polyurethanes are used in shoes, automobiles, airplanes, bushings, gaskets, adhesives, carpeting, spandex fibers, housing for electronics and the like.

In another embodiment, the present invention relates to a method of producing a product. An epoxidized fatty acid ester is reacted with an alcohol to open an epoxy ring of the ester, using a salt of tetrafluoroboric acid as a catalyst in the reaction, to thereby produce a polyol. Then the polyol is used to produce a desired product. For example, the product may be selected from polyurethanes, coatings, adhesives, sealants, elastomers and lubricants. The product can be produced using the polyol in combination with other suitable product components, and using suitable manufacturing techniques. For example, methods and compositions for producing soybean oil based lubricants are disclosed in US 2012/0129746, published May 24, 2012, which is incorporated by reference herein.

### EXAMPLES

### Example 1

### Ring Opening of Epoxidized Algal Oil with Fluoroboric Acid or Copper Tetrafluoroborate Catalyst

To a 3 L 3-neck round bottom flask equipped with a mechanical stirrer, thermocouple, addition funnel, heating mantle and Argon gas inlet was charged 500 mL of methanol and 21.20 g of 48% fluoroboric acid. The mixture was stirred and heated to 40° C. Over a 1.5 hour time period, a mixture of 915.60 g of epoxidized algal oil and 400 mL of chloroform was added dropwise to the reaction flask. An additional 120 mL of chloroform was used to rinse the remaining oil into the reaction flask. After 1 hour, the reaction mixture was sampled and oxirane conversion determined to be complete by proton NMR. To the reaction flask, 11 g of sodium bicarbonate was added with stirring to neutralize the acid. A majority of the solvent was removed under reduced pressure at 40° C. The polyol was then dissolved in 1 L of ethyl acetate and extracted with 3×100 mL of a 20% potassium carbonate solution. The organic layer was then dried over magnesium sulfate, filtered and solvent removed at 80° C. under reduced pressure to yield 951.71 g (95.7% yield) of the methyl ether alcohol. The hydroxyl value was determined by NMR to be 146 mg KOH/g.

The product was then combined with a similarly prepared batch (1955 g, 93.9% yield, HVNMR=145) to constitute the final methyl ether alcohol (HVNMR=145 and HVTitration=145). Alternatively, the fluoroboric acid can be replaced with copper tetrafluoroborate. This replacement was found to result in a number of advantages including allowing for the addition of a full charge of reagents instead of dropwise addition, less diglyceride formation, eliminating need for acid neutralization and resulting salt removal, extraction of catalyst with water/brine wash, and diminished reaction exotherm allowing for less solvent use. The ethyl acetate in the workup can also be replaced with methyl acetate. This replacement was found to result in less emulsification and easier separation. Additionally removal of the solvent was not necessary and can be carried on to the urethane condensation reaction.

### Example 2

### Ring Opening of Epoxidized Soybean Oil with Iron Tetrafluoroborate Catalyst

148.45 g of epoxidized soybean oil (ESO) and 61 mL methanol were placed into a 3 neck round bottom flask containing a thermocouple, condenser, and magnetic stirrer. The mixture was heated to 35 C using a temperature controller, internal thermocouple, and heating mantle. 3.89 g of iron (II) tetrafluoroborate were dissolved into 60 mL methanol and added to the ESO mixture at 1:17 PM. The cloudy mixture immediately exotherms. At 1:19 PM temperature was 59.0 C and the mixture begins refluxing at 1:20 PM. The mixture turns clear at 1:21 PM and temperature is 69.7 C (set point is 60 C). At 1:25 Pm the mixture is light yellow and turbid at 63.7 C. Took 1H NMR sample at 1:55 PM. Reaction was complete.

1. A ring opening method comprising reacting an epoxidized fatty acid ester with an alcohol to open an epoxy ring of the ester, using a salt of tetrafluoroboric acid as a catalyst in the reaction.

2. The method of claim 1 wherein the salt is a metal salt.

3. The method of claim 2 wherein the metal is copper or iron.

4. The method of claim 1 wherein the fatty acid ester is a triglyceride.

5. The method of claim 4 wherein the triglyceride is selected from the group consisting of vegetable oils, algal oils, animal fats and combinations thereof.

6. The method of claim 5 wherein the triglyceride is selected from the group consisting of vegetable oils, animal fats and combinations thereof.

7. The method of claim 4 wherein the triglyceride excludes algal oils.

8. The method of claim 4 wherein the triglyceride comprises soybean oil.

9. The method of claim 1 wherein the alcohol is a monohydric alcohol having from one to six carbon atoms.

10. The method of claim 1 wherein the alcohol is a polyol having two or less hydroxyl groups.

11. The method of claim 1 wherein the product of the ring opening reaction is a polyol.

12. The method of claim 10 wherein the polyol has a hydroxyl value within a range from about 75 to about 400.

13. The method of claim 1 wherein the product of the ring opening reaction is subjected to a further reaction to produce a polyol.

14. The method of claim 1 further comprising a step of water washing the product to remove catalyst without neutralization.

15. A method of producing a polyurethane comprising:
reacting an epoxidized fatty acid ester with an alcohol to open an epoxy ring of the ester, using a salt of tetrafluoroboric acid as a catalyst in the reaction, to produce a polyol; and
reacting the polyol with an isocyanate to produce a polyurethane.

16. The method of claim 15 wherein the salt is a metal salt.

17. The method of claim 15 wherein the fatty acid ester is a triglyceride.

18. The method of claim 17 wherein the triglyceride is selected from the group consisting of vegetable oils, animal fats and combinations thereof.

19. The method of claim 15 comprising an additional step, before the ring opening reaction, of subjecting the fatty acid ester to an epoxidation reaction to produce the epoxidized fatty acid ester.

20. A method of producing a product comprising:
reacting an epoxidized fatty acid ester with an alcohol to open an epoxy ring of the ester, using a salt of tetrafluoroboric acid as a catalyst in the reaction, to produce a polyol; and
using the polyol to produce a product selected from polyurethanes, coatings, adhesives, sealants, elastomers and lubricants.
